(19) United States
(12) Patent Application Publication
(10) Pub. No.: US 2017/0119265 A1
(43) Pub. Date: May 4, 2017

(54) System and Method for using Demographic Data to Derive a Pulse Wave Velocity-Blood Pressure Transform

(71) Applicant: Sharp Laboratories of America (SLA), Inc., Camas, WA (US)

(72) Inventor: Fredrick Hill, Portland, OR (US)

(21) Appl. No.: 15/175,558

(22) Filed: Jun. 7, 2016

Related U.S. Application Data
(63) Continuation-in-part of application No. 14/983,348, filed on Dec. 29, 2015.
Application No. 14/983,348 is a continuation-in-part of application No. 14/932,019, filed on Nov. 4, 2015.

(51) Int. Cl. A61B 5/021; A61B 5/0402; A61B 5/00

(52) U.S. Cl. CPC A61B 5/02125; A61B 5/7225; A61B 5/0402; A61B 5/7278; A61B 5/7475; A61B 2560/0238; A61B 2560/0475

(57) ABSTRACT

A system and method are provided for using demographic data to derive a PWV-BP transform. The method provides a PWV measurement device with a non-transitory memory, processor, and a calibration application for supplying pseudo-calibrated PWV values. The method loads into the memory a first database of information cross-referencing age compared to central-aortic PWV-BP transforms, a second database of information cross-referencing age and gender compared to systolic and diastolic blood pressure, and a third database of information cross-referencing age as compared to whole-arm PWV. Whole-arm PWV measures a distance between a superasternal notch and index finger, divided by a transit time of an arterial pulse from to heart to the index finger tip. After accepting age and gender data from a first user, the calibration application interpolates the information from the first, second, and third databases, and derives a pseudo-calibrated whole-arm PWV-BP transform for the first user.

### RELATED APPLICATIONS

This application incorporates by reference an application entitled, PULSE WAVE VELOCITY-TO-BLOOD PRESSURE CALIBRATION PROMPTING, invented by Fredrick Hill, U.S. Ser. No. 14/983,348, filed Dec. 29, 2015, Attorney Docket No. SLA3577.

This application incorporates by reference an application entitled, SYSTEM AND METHOD FOR DERIVING A PULSE WAVE VELOCITY-BLOOD PRESSURE TRANSFORM, invented by Fredrick Hill, U.S. Ser. No. 14/932,019, filed Nov. 4, 2015, Attorney Docket No. SLA3572.

### BACKGROUND OF THE INVENTION

1. Field of the Invention

This invention generally relates to blood pressure measurement and, more particularly, to a system and method for deriving a pulse wave velocity-to-blood pressure transform parameterized by the demographic features of a patient.

2. Description of the Related Art

In recent years, consensus has developed that a strong correlation exists between arterial pulse wave velocity (PWV) and systolic and diastolic blood pressure. The PWV is derived from the length of an arterial segment and the time required, on average, for the arterial pulse to traverse that distance. More explicitly, a PWV measurement involves a combination of simultaneous electrocardiography (ECG or EKG) and photoplethysmography (PPG) measurements. Electrocardiography is the process of recording the electrical activity of the heart over a period of time using electrodes placed on a patient's body. These electrodes detect the tiny electrical changes on the skin that arise from the heart muscle depolarizing during each heartbeat. During each heartbeat, a healthy heart has an orderly progression of depolarization that starts with pacemaker cells in the sinoatrial node, spreads out through the atrium, passes through the atrioventricular node down into the bundle of His and into the Purkinje fibers spreading down and to the left throughout the ventricles. This orderly pattern of depolarization gives rise to the characteristic ECG tracing.

Photoplethysmography is a method of measuring the perfusion of blood to the dermis and subcutaneous tissue by illuminating the tissue at the surface and observing variations of the light. With each cardiac cycle the heart pumps blood to the periphery. The change in blood volume caused by the pressure pulse of the cardiac cycle is detected by illuminating the skin with a light-emitting diode (LED) and measuring the amount of light either transmitted or reflected to a photodiode, The resulting waveform characterizes the relative blood volume of the tissue over time.

PWV-based blood pressure (PWV-BP) addresses many limitations of the oscillometric and auscultatory methods. It requires no arterial compression, no cuff, and no recovery interval. A measurement can be formed on every arterial pulse and integrated over time to reduce measurement uncertainty. In some modalities, it is possible to collect the measurement in a worn device and continuously update the blood pressure estimate. PWV is proportional to blood pressure according to a PWV-BP transform which varies from person to person. However, PWV-based blood pressure requires a transform from PWV to blood pressure and that transform varies from patient to patient, over populations, and over time. Some PWB-BP designs require calibration—the PWV-BP transform is derived by taking multiple simultaneous measurements of PWV and BP—and fitting the transform curve to those calibrations. Other approaches may eschew calibration entirely and base the transform on population norms.

A key consideration in PWV-BP is that the transform differs significantly between patients. Age-related differences in the PWV-BP transform were described in a large study (n=11,092) reported in the European Heart Journal [1]. The study reported mean PWV-BP slope by age decade. The magnitude differences in PWV-BP transform slope between adjacent age decades averaged 13.8%. Given that individuals do not age uniformly, a transform tailored to the individual is necessary to accurately transform PWV to BP. The adjustment of the transform to the individual may be accomplished through calibration.

PWV has many modalities. It is typically measured differentially between the femoral and carotid arteries. However, a measurement can be derived from the time difference between the ECG R-wave and the PPG pulse measured at the index finger. In this measurement, the ECG signal is corrected for PEP [4,5] and latency in the signal path. The time interval between the R-wave and the foot of the PPG pulse is measured repeatedly, filtered to remove outliers, and then averaged to estimate the mean pulse transit time. The distance between the patient's suprasternal notch and tip of the index finger is divided by the pulse transit time to yield the PWV. As such, the PWV of interest here might be described as “whole-arm” PWV.

A PWV-BP calibration measurement typically consists of reference systolic and diastolic blood pressures and a PWV. With multiple calibrations, it is possible to adjust a patient's PWV-BP transform by fitting it to the calibration data. However, it is not always possible or convenient to simultaneously take both PB and PWV measurements.

It would be advantageous if an accurate PWV-BP transform could be obtained from demographic data, without the requirement of calibration measurements.
1. European Heart Journal, Volume 31, Issue 19, pp. 2338 - 2350, June 2010.
2. National Health Statistics Report, Number 35, Mar. 25, 2011.
3. Fulton, J. S., B. A. McSwiney, “The Pulse Wave Velocity and Extensibility of the Brachial Artery in Man”, Wiley Online Library, June 1930.
4. Hodges, M., et al, “Left Ventricular Projection Period and Ejection Time in Patients with Acute Myocardial Infarction”, Circulation, vol. XLV, May 1972.
5. Zhang, G,, et al., “Assessing the Challenges of a Pulse Wave Velocity Based Blood Pressure Measurement in Surgical Patients”, IEEE EMBS, 2014: 574-577.

### SUMMARY OF THE INVENTION

Pulse-wave Velocity Blood Pressure (PWV-BP) is a method for deriving a blood pressure measurement from a measurement of arterial pulse wave velocity (PWV). The PWV is derived from the length of an arterial segment and the time required, on average, for the arterial pulse to traverse that distance. PWV is proportional to blood pressure according to a PWV-BP transform which varies from person to person. The PWV-BP transform can be derived by taking multiple calibrations—i.e., simultaneous measurements of PWV and BP—and fitting the transform curve to those calibrations. While calibration is necessary for optimal performance, it is often desirable to support operation of a PWV-BP device in uncalibrated mode. The ability to operate, even with reduced accuracy, during this calibration phase is key to the viability of a PWV-BP product. The method described here derives normal values of PWV, blood pressure, and transform slope from the patient's demographic data and uses those values as an adjunct for calibration measurements, allowing PWV-BP device operation prior to the initial calibration. The demographic data may include age and gender (since these are significant factors), and transform whole-arm PWV (since that is the modality of interest here).

Accordingly, a method is provided for using demographic data to derive a PWV-BP transform. The method provides a PWV measurement device with a non-transitory memory, processor, and a calibration application for supplying pseudocalibrated PWV values. The method loads into the memory a first database of information cross-referencing age compared to central-aortic PWV-BP transforms, a second database of information cross-referencing age and gender compared to systolic and diastolic blood pressure, and a third database of information cross-referencing age as compared to whole-arm PWV. Whole-arm PWV measures a distance between a superasternal notch and index finger, divided by a transit time of an arterial pulse from to heart to the index finger tip. After accepting age and gender data from a first user, the calibration application interpolates the information from the first, second, and third databases, and derives a pseudo-calibrated whole-arm PWV-BP transform for the first user.

In one aspect, interpolating the information from the first, second, and third databases includes the following substeps, A systolic blood pressure transform (SBPXfrm) is determined incorporating a systolic blood pressure (SBP) derived from the second database, a whole-arm PWV (PWV_{Arm}) derived from the third database, a central aortic PWV (PWV_{Central}) derived from the first database, and a transform slope (Slope_{Central}) derived from the first database. A diastolic blood pressure transform (DBPXfrm) is determined incorporating the diastolic blood pressure (DBP) derived from the second database, the PWV_{Arm} derived from the third database, the PWV_{Central} derived from the first database, and the Slope_{Central} derived from the first database. Optionally, a mean blood pressure transform (MBPXfrm) is determined incorporating a mean blood pressure (MBP) derived from the second database, the PWV_{Arm} derived from the third database, the PWV_{Central} derived from the first database, and the Slope_{Central} derived from the first database.

For example, the SBPXfrm is determined by:

fitting the SBP data derived from the second database into quadratic curves parameterized by age, for each gender;

fitting the PWV_{Arm} derived from the third database into a quadratic curve parameterized by age;

fitting the PWV_{Central} derived from the first database into a quadratic curve parameterized by age; and,

fitting the Slope_{Central} derived from the first database into a quadratic curve parameterized by age.

Then, a SBPXfrm intercept point and slope are found as follows:

$$SBPXfrm = [ {b}_{s} , {m}_{s} ] wherein$$

$${m}_{s} = {Slope}_{Central} ( age ) \times \frac{{PWV}_{Arm} ( age )}{{PWV}_{Central} ( age )}$$

$${b}_{s} = SBP ( age , gender ) - {m}_{s} \times {PWV}_{Arm} ( age ) .$$

The DBPXfrm and MBPXfrm are found in a similar manner.

As a result, after measuring the whole-arm PWV of the first user, the pseudo-calibrated whole-arm PWV-BP transform can he used to derive (without calibration measurements) a blood pressure associated with the first user whole-arm PWV measurement. In one aspect, the pseudo-calibrated PWV-BP transform can be more precisely based upon a current date and the first user's date of birth.

Additional details of the above-described method and a system using demographic data to derive a PWV-BP transform are presented below.

### BRIEF DESCRIPTION OF THE DRAWINGS

FIG. 1 is a schematic block diagram depicting a system using demographic data to derive a pulse wave velocity-blood pressure (PWV-BP) transform.

FIG. 2 is a graph depicting quadratic fits between systolic blood pressure and age for male, female, and general populations (prior art).

FIG. 3 is a graph depicting quadratic fits between normal central and whole-arm PWV, expressed in meters per second (m/s), and age (prior art).

FIG. 4 is a graph depicting a linear fit between mean blood pressure and central aortic PWV, by age decades (prior art).

FIG. 5 is a graph depicting a quadratic fit between normal central aortic PWV-BP slope and age, with the corrected whole-arm PWV-BP slope curve shown.

FIGS. 6A through 6E are a collection of linked flowcharts illustrating a method for using demographic data to derive a PWV-BP transform.

FIG. 7 illustrates the application of equations 1 through 7 to the calibration of the pseudo-calibrated SBP transform.

### DETAILED DESCRIPTION

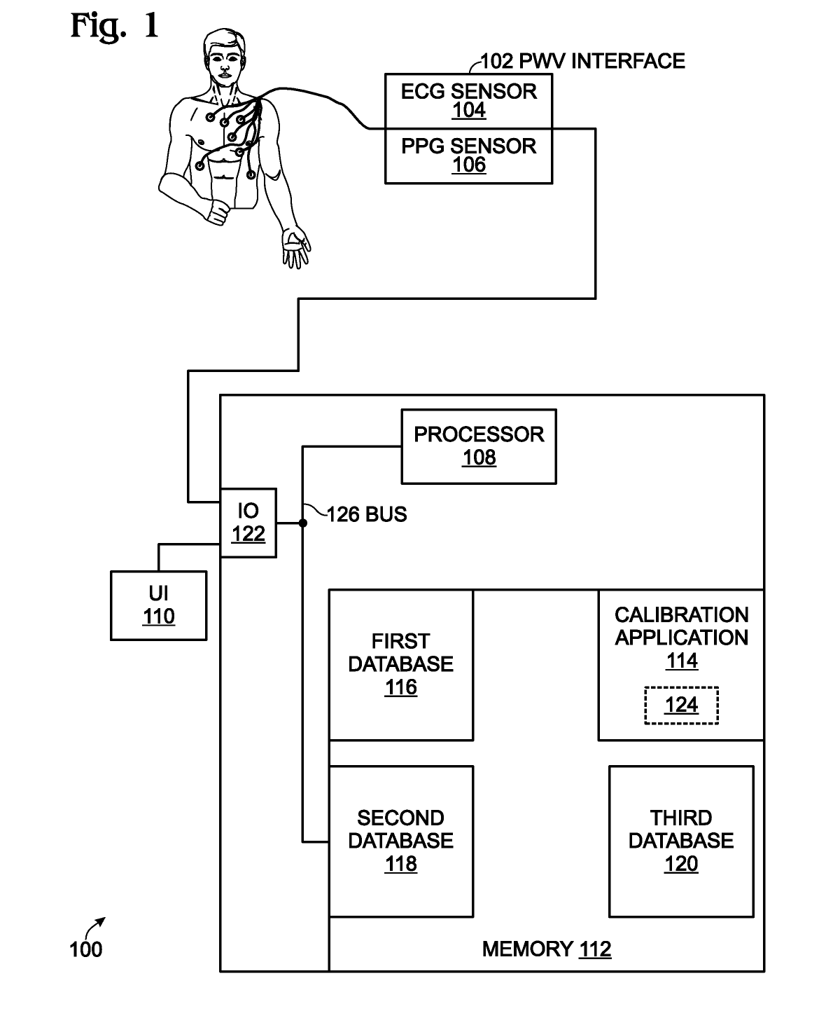

FIG. 1 is a schematic block diagram depicting a system using demographic data to derive a pulse wave velocity-blood pressure (PWV-BP) transform. The system 100 comprises a PWV measurement interface 102 comprising an electrocardiogram (ECG) sensor 104 and a photoplethysmography (PPG) sensor 108 for respectively measuring first user ECG and PPG signals. Typically, the PPG sensor 106 comprises a light emission device and a light sensing device (not shown) for detecting changes in optical transmittance of an illuminated test subject body. Typically, the ECG sensor 104 comprises at least two electrodes (not shown).

The system 100 further comprises a processor 108 and a user interface (UI) 110 to accept age information from the first user, and to supply a pseudo-calibrated blood pressure (BP) value. The system 100 also comprises a non-transitory memory 112. A calibration application 114 is embedded in the memory 112 and enabled as a sequence of processor executable steps. The calibration application 114 accepts the ECG and PPG signals, the first user age, and information interpolated from a first database, a second database, and a third database, to calculate the pseudo-calibrated blood pressure BP value for the first user. In one aspect, the UI 110 accepts first user gender information, and the calibration application 114 calculates the pseudo-calibrated BP value in response to the first user gender (as well as age).

In another aspect, the UI 110 accepts a first user specific date of birth, and the calibration application 114 calculates the pseudo-calibrated BP value based upon a current date, the first user's date of birth, and quadratic models of PWV-BP transform slope, PWV, and blood pressure for the first user's date of birth.

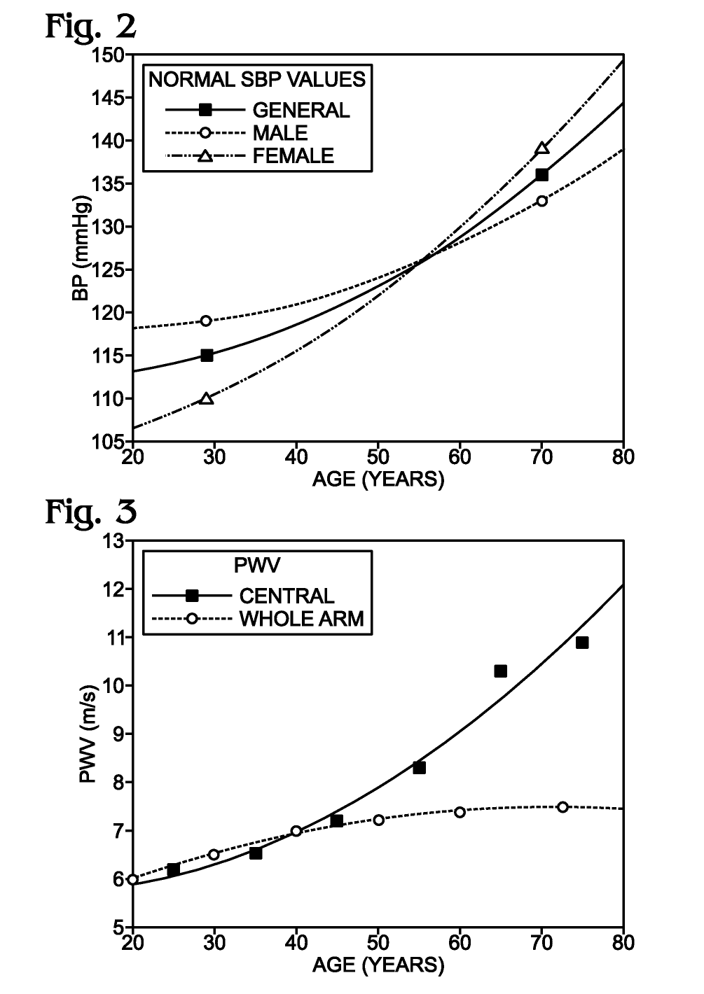

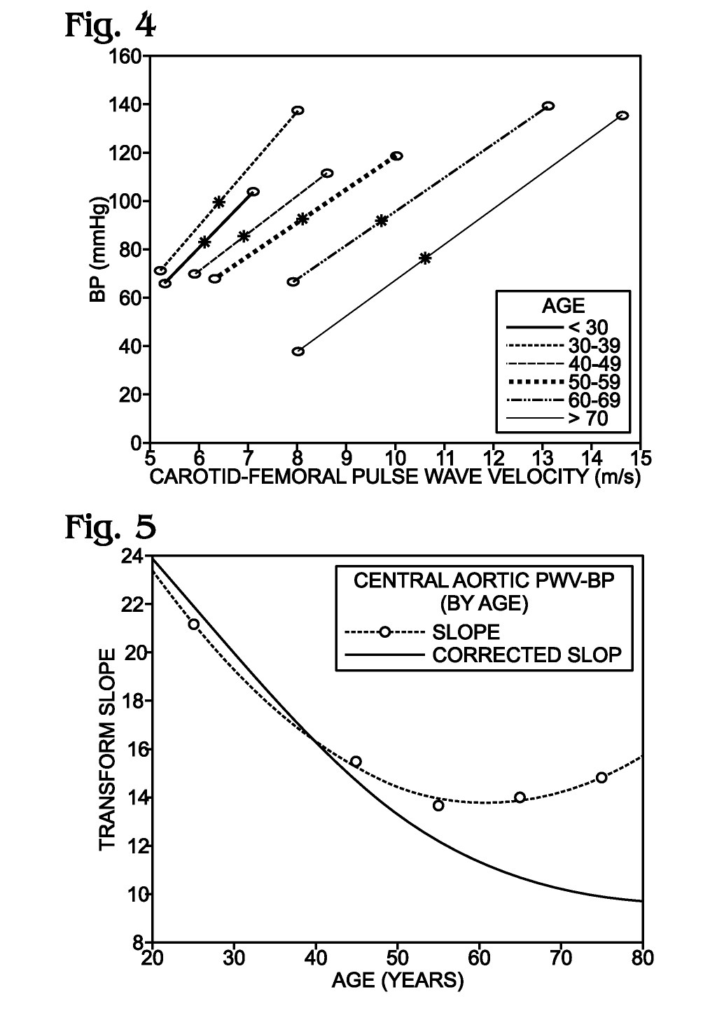

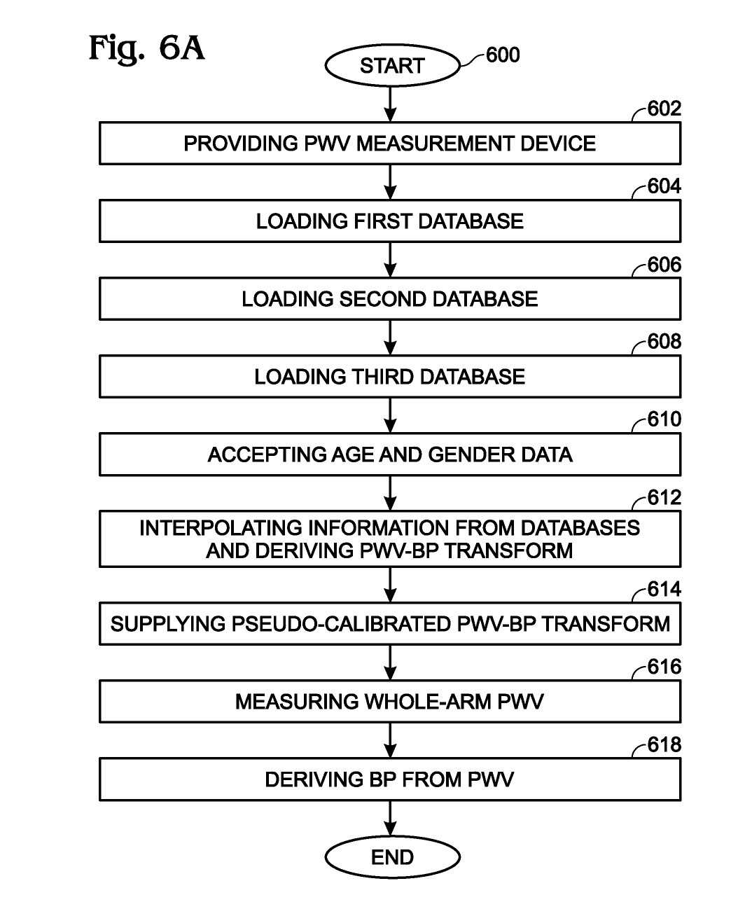

The first database 116 of information cross-references age compared to central-aortic PWV-BP transforms, see FIG. 4. The second database 118 of information cross-references age and gender compared to systolic and diastolic blood pressure, see FIG. 2. The third database 120 of information cross-references age as compared to whole-arm PWV, see FIG. 3. As defined herein, whole-arm PWV measures a distance between a superasternal notch and index finger, divided by a transit time of an arterial pulse from to heart to the index finger tip. In one aspect, the first 116, second 118, and third 120 databases reside in the memory 112, as shown, which permits the calibration application 114 to calculate transforms. Alternatively, the databases may reside in a remote memory (not shown) in contact with calibration application via input/output (IO) port 122. As another alternative, the transforms and/or quadratic polynomials derived from the first database 116, second database 118, and third database 120 are pre-calculated and a module 124 (in phantom) of the calibration application 114.

If the transforms are not pre-calculated, the calibration application 114 may determine a systolic blood pressure transform (SBPXfrm) by incorporating the systolic blood pressure (SBP) derived from the second database, the whole-arm PWV (PWV_{Arm}) derived from the third database, the central aortic PWV (PWV_{Central}) derived from the first database, and a transform slope (Slope_{Central}) derived from the first database. Similarly, the calibration application 114 may determine a diastolic blood pressure transform (DBPXfrm) incorporating the diastolic blood pressure (DBP) derived from the second database, the PWV_{Arm} derived from the third database, the PWV_{Central} derived from the first database, and the Slope_{Central} derived from the first database. Optionally, the calibration application 114 may determine a mean blood pressure transform (MBPXfrm) incorporating a mean blood pressure (MBP) derived from the second database, the PWV_{Arm} derived from the third database, the PWV_{Central} derived from the first database, and the Slope_{Central} derived from the first database.

More explicitly, the calibration application 114 determines the SBPXfrm by fitting the SBP data derived from the second database into quadratic curves parameterized by age, for each gender. The PWV_{Arm} derived from the third database is fit into a quadratic curve parameterized by age. The PWV_{Central} derived from the first database is fit into a quadratic curve parameterized by age, and the Slope_{Central} derived from the first database is fit into a quadratic curve parameterized by age.

Next, the calibration application 114 determines a SBPXfrm intercept point and slope as follows:

$$SBPXfrm = [ {b}_{s} , {m}_{s} ] wherein$$

$${m}_{s} = {Slope}_{Central} ( age ) \times \frac{{PWV}_{Arm} ( age )}{{PWV}_{Central} ( age )}$$

$${b}_{s} = SBP ( age , gender ) - {m}_{S} \times {PWV}_{Arm} ( age ) ;$$

wherein Slope_{Central}(age) is a quadratic model of central aortic PWV-BP slope as a function of age:

wherein PWV_{Arm}(age) is a quadratic model of whole-arm PWV as a function of age;

wherein PWV_{Central}(age) is a quadratic model of central aortic PWV as a function of age and,

wherein SBP(age, gender) is a quadratic model of systolic blood pressure as a function of age and gender.

The calibration application determines the DBPXfrm in a similar manner by fitting the DBF derived from the second database into a quadratic curve parameterized by age, for each gender. The PWV Arm derived from the third database is fit into a quadratic curve parameterized by age. The PWV_{Central} derived from the first database is fit into a quadratic curve parameterized by age, and the Slope_{Central} derived from the first database is fit into a quadratic curve parameterized by age.

The calibration application 114 then determines a DBPXfrm intercept point and slope as follows:

$$DBPXfrm = [ {b}_{d} , {m}_{d} ] . wherein$$

$${m}_{d} = {Slope}_{Central} ( age ) \times \frac{{PWV}_{Arm} ( age )}{{PWV}_{Central} ( age )}$$

$${b}_{d} = SBP ( age , gender ) - {m}_{d} \times {PWV}_{Arm} ( age ) ;$$

wherein Slope_{Central}(age) is a quadratic model of central aortic PWV-BP slope as a function of age:

wherein PWV_{Arm}(age) is a quadratic model of whole-arm PWV as a function of age;

wherein PWV_{Central}(age) is a quadratic model of central aortic PWV as a function of age; and,

wherein DBP(age, gender) is a quadratic model of systolic blood pressure as a function of age and gender.

Likewise, the calibration application 114 determines the MBPXfrm by fitting the MBP derived from the second database into quadratic curves parameterized by age, for each gender. The PWV_{Arm} derived from the third database is fit into a quadratic curve parameterized by age. The PWV_{Central} derived from the first database is fit into a quadratic curve parameterized by age, and the Slope_{Central} derived from the first database is fit into a quadratic curve parameterized by age.

The calibration application 114 determines a MBPXfrm intercept point and slope as follows:

$$MBPXfrm = [ {b}_{m} , {m}_{m} ] \text{:} wherein$$

$${m}_{m} = {Slope}_{Central} ( age ) \times \frac{{PWV}_{Arm} ( age )}{{PWV}_{Central} ( age )}$$

$${b}_{m} = MBP ( age , gender ) - {m}_{m} \times {PWV}_{Arm} ( age ) ;$$

wherein Slope_{Central}(age) is a quadratic model of central aortic PWV-BP slope as a function of age:

wherein PWV_{Arm}(age) is a quadratic model of whole-arm PWV as a function of age;

wherein PWV_{Central}(age) is a quadratic model of central aortic PWV as a function of age; and,

wherein MBP(age, gender) is a quadratic model of systolic blood pressure as a function of age and gender.

The system 100 may be understood to be a computing device. As such it may include a communications bus 126 connected to the IO port 122, processor 108, memory 111, and UI 110. The communication bus 126 may, for example, be a Serial Peripheral Interface (SPI), an Inter-Integrated Circuit (I2C), a Universal Asynchronous Receiver/Transmitter (UART), and/or any other suitable bus or network. Although the drawing implies that the components of the system 100 are collocated in the same device, in some aspects various components may be located outside the device, communicating with other components via a wired or wireless connection.

The memory 112 may include a main memory, a random access memory (RAM), or other dynamic storage devices. These memories may also be referred to as a computer-readable medium. Such a medium may take many forms, including but not limited to, non-volatile media, volatile media, and transmission media. Non-volatile media includes, for example, optical or magnetic disks. Volatile media includes dynamic memory. Common forms of computer-readable media include, for example, a floppy disk, a flexible disk, hard disk, magnetic tape, or any other magnetic medium, a CD-ROM, any other optical medium, a RAM, a PROM, and EPROM, a FLASH-EPROM, any other memory chip or cartridge, or any other medium from which a computer can read. The execution of the sequences of instructions contained in a computer-readable medium may cause the processor 108 to perform some of the steps of determining the PWV-BP transform. The practical implementation of such a computer system would be well known to one with skill in the art. In one aspect, the processor 108 is an ARM processor using a reduced instruction set computing (RISC) architecture.

The IO port 126 may incorporate a modem, an Ethernet card, or any other appropriate data communications device such as USB. The physical communication links may be optical, wired, or wireless. The user interface 110 may incorporate a keypad or a cursor control device such as a mouse, touchpad, touchscreen, trackball, stylus, or cursor direction keys.

The system 100 may provide a direct connection to a remote server via a direct link to a network, such as the Internet. Connection may be provided through, for example, a local area network (such as an Ethernet network), a personal area network, a wide area network, a private network (e.g., a virtual private network), a telephone or cable network, a cellular telephone connection, a satellite data connection, or any other suitable connection.

FIG. 2 is a graph depicting quadratic fits between systolic blood pressure and age for male, female, and general populations (prior art). The quadratic equations behind these curves fit to normal systolic BP to age and gender.

FIG. 3 is a graph depicting quadratic fits between normal central and whole-arm PWV, expressed in meters per second (m/s), and age (prior art).

FIG. 4 is a graph depicting a linear fit between mean blood pressure and central aortic PWV, by age decades (prior art).

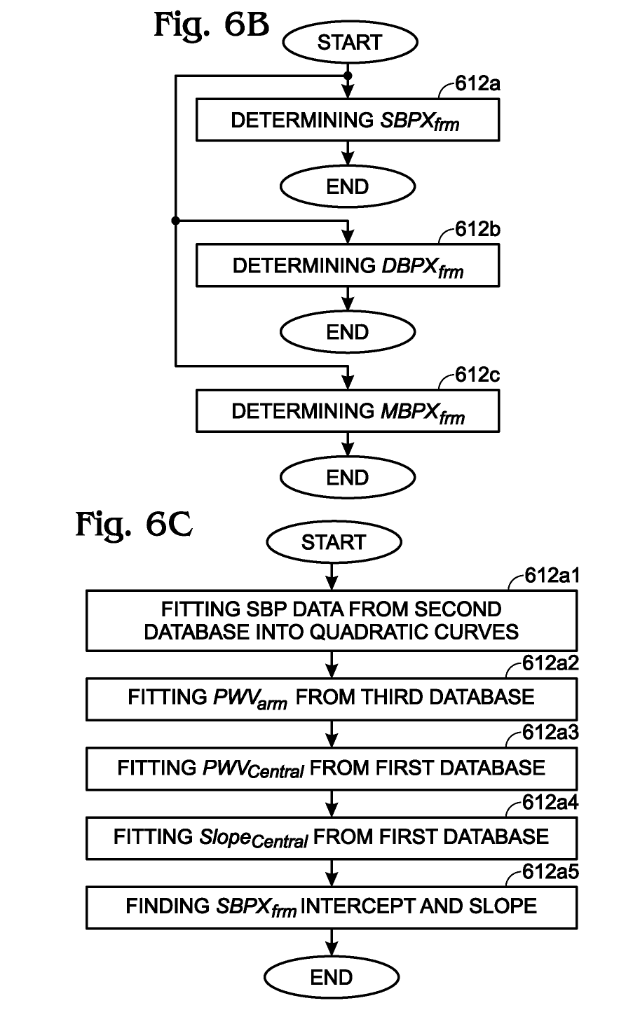

FIG. 5 is a graph depicting a quadratic fit between normal central aortic PWV-BP slope values and age, with the corrected whole-arm PWV-BP slope curve shown.

To establish the transform slope, a quadratic is fit to the transform slopes from [1] shown in FIG. 4. That quadratic expresses the normal central aortic PWV-BP slope as a function of age and is shown in FIG. 5 as the line marked with small circles. To derive normal whole-arm PWV-BP slope as a function of age, that quadratic is multiplied by the ratio of the whole arm and central aortic curves to yield a curve as shown in the lower unmarked line in FIG. 5. In more formal terms, two demographic parameters, age and gender are used to produce the linear transform:

SBPXfrm(age, gender)≡[b,m] (1)

where b is the y-intercept and m is the slope of the transform. Quadratic transforms are derived representing the normal values of blood pressure, central aortic PWV, whole-arm PWV, and central aortic BP transform slope:

PWV_{Central}(age)=0.001196×age²−0.016786×age+5.732589 (2)

PWV_{Arm}(age)=−0.000567×age²+ 0.080763×age+4.612566 (3)

Slope_{Central}(age)=0.005641×age²−0.692275×age+34.999349 (4)

These transforms were derived by curve fits to the data provided in [1], [2], and [3]. An additional transform is provided to select the appropriate SBP transform by gender.

SBP(age, gender)=(gender==male)?SBPₘₐₗₑ(age):SBP_{female}(age) (5)

Definition (1) is further defined as follows, for parameters PWV, age, and gender:

$$\begin{matrix}m = {Slope}_{Central} ( age ) \times \frac{{PWV}_{Arm} ( age )}{{PWV}_{Central} ( age )} & ( 6 ) \\ b = SBP ( age , gender ) - m \times {PWV}_{Arm} ( age ) & ( 7 )\end{matrix}$$

In the above, for the sake of brevity, only systolic blood pressure is considered. Diastolic and mean pressures differ only in the transform coefficients.

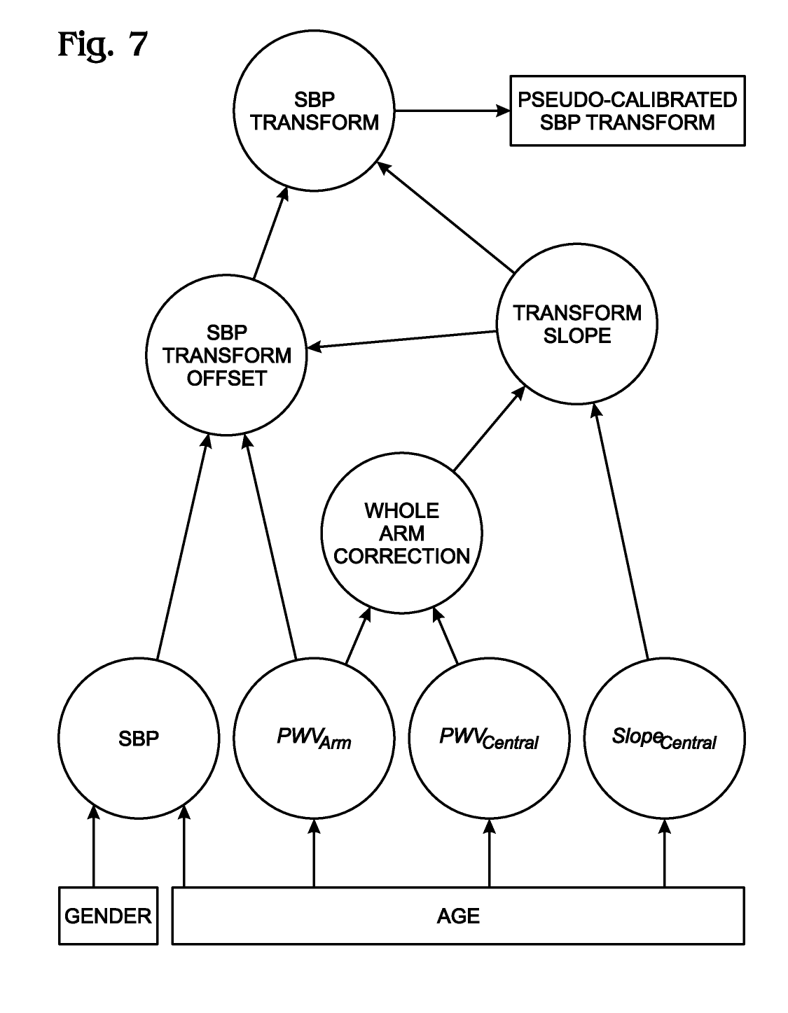

FIG. 7 illustrates the application of equations 1 through 7 to the calibration of the pseudo-calibrated SBP transform. This method uses a pseudo-calibration point derived from normal BP and PWV values for the patient's age and gender. It derives a transform slope from population PWV-BP curves transformed to the whole-arm PWV of interest here. Because the quantities are derived from normal values, in the absence of actual calibration data this method represents a reasonable guess of the patient's PWV-BP state. Over a large number of trials, this transform should yield a low bias estimate of blood pressure. Of course, people rarely reflect their normal values of blood pressure, PWV, and PWV-BP transform slope. The error given by this method is likely larger than if those values are measured. Nonetheless, this method provides a “ballpark” estimate that allows relative comparison of blood pressure values over time. It meets the need for uncalibrated PWV-BP measurement in a PWB-BP device prior to initial calibration.

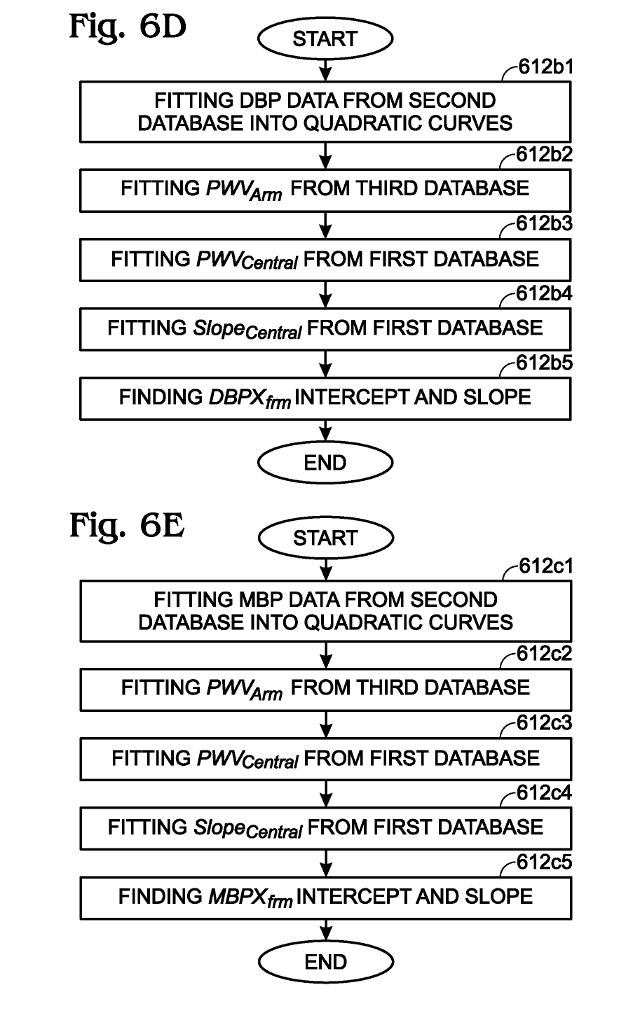

FIGS. 6A through 6E are a collection of linked flowcharts illustrating a method for using demographic data to derive a PWV-BP transform. Although the method is depicted as a sequence of numbered steps for clarity, the numbering does not necessarily dictate the order of the steps. It should be understood that some of these steps may be skipped, performed in parallel, or performed without the requirement of maintaining a strict order of sequence. Generally however, the method follows the numeric order of the depicted steps. The method starts in FIG. 6A at Step 600.

Step 602 provides a PWV measurement device comprising a non-transitory memory, processor, and a calibration application enabled as a sequence of processor executable steps for providing pseudo-calibrated PWV values. Step 804 loads a first database into the memory cross-referencing age and central-aortic PWV-BP transforms. Step 806 loads a second database into the memory cross-referencing age and gender with systolic and diastolic blood pressure. Step 608 loads a third database into the memory cross-referencing age and whole-arm PWV. As noted above, whole-arm PWV measures a distance between a superasternal notch and index finger, divided by a transit time of an arterial pulse from to heart to the index finger tip.

Step 610 accepts age and gender data from a first user. In Step 612 the calibration application interpolates the information from the first, second, and third databases, and derives a pseudo-calibrated whole-arm PWV-BP transform. Step 614 supplies the pseudo-calibrated whole-arm PWV-BP transform for the first user. Step 616 measures the whole-arm PWV of the first user. Step 618 uses the pseudo-calibrated whole-arm PWV-BP transform to derive (without making actual measurements to correlate PWV to PB) a blood pressure associated with the first user whole-arm PWV measurement.

In one aspect, the first database cross-references patient age to central-aortic PWV-BP transforms, the second database cross-references patient age to SBP and DBP, and the third database cross-references age to whole-arm PWV, Then, accepting the age data from the first user in Step 610 includes accepting a specific date of birth, and Step 612 derives a pseudo-calibrated PWV-BP transform based upon a current date and the first user's date of birth.

In one aspect, interpolating the information from the first, second, and third databases in Step 612 includes substeps, see FIG. 6B. Step 612a determines a systolic blood pressure transform (SBPXfrm) incorporating the systolic blood pressure (SBP) derived from the second database, the whole-arm PWV (PWV_{Arm}) derived from the third database, the central aortic PWV (PWV_{Central}) derived from the first database, and a transform slope (Slope_{Central}) derived from the first database. Step 812b determines a diastolic blood pressure transform (DBPXfrm) incorporating the diastolic blood pressure (DBP) derived from the second database, the PWV_{Arm} derived from the third database, the PWV_{Central} derived from the first database, and the Slope_{Central} derived from the first database. Step 612c may determine a mean blood pressure transform (MBPXfrm) incorporating a mean blood pressure (MBP) derived from the second database, the PWV_{Arm} derived from the third database, the PWV central derived from the first database, and the Slope_{Central} derived, from the first database.

Determining the SBPXfrm in Step 612a includes additional substeps, see FIG. 6C. Step 612a1 fits the SBP data derived from the second database into quadratic curves parameterized by age, for each gender. Step 612a2 fits the PWV_{Arm} derived from the third database into a quadratic curve parameterized by age. Step 612a3 fits the PWV_{Central} derived from the first database into a quadratic curve parameterized by age. Step 612a4 fits the Slope_{Central} derived from the first database into a quadratic curve parameterized by age. In Step 612a5 the SBPXfrm intercept point (bₛ) and slope (mₛ) are found as follows:

$$SBPXfrm = [ {b}_{s} , {m}_{s} ] wherein$$

$${m}_{s} = {Slope}_{Central} ( age ) \times \frac{{PWV}_{Arm} ( age )}{{PWV}_{Central} ( age )}$$

$${b}_{s} = SBP ( age , gender ) - {m}_{s} \times {PWV}_{Arm} ( age ) .$$

Determining the DBPXfrm in Step 612b includes the following substeps, see FIG. 6D. Step 612b1 fits the DBF derived from the second database into a quadratic curve parameterized by age, for each gender. Step 612b2 fits the PWV_{Arm} derived from the third database into a quadratic curve parameterized by age. Step 612b3 fits the PWV_{Central} derived from the first database into a quadratic curve parameterized by age. Step 612b4 fits the Slope_{Central} derived from the first database into a quadratic curve parameterized by age. Note: Steps 612b2 through 612b4 are the same as Steps 612a2 through 612a4, and need only be performed once for SBP, DBP, and MBP. Step 612b5 finds the DBPXfrm intercept point (b_{d}) and slope (m_{d}) as follows:

$$DBPXfrm = [ {b}_{d} , {m}_{d} ] . wherein$$

$${m}_{d} = {Slope}_{Central} ( age ) \times \frac{{PWV}_{Arm} ( age )}{{PWV}_{Central} ( age )}$$

$${b}_{d} = DBP ( age , gender ) - {m}_{d} \times {PWV}_{Arm} ( age ) ;$$

Determining the MBPXfrm includes additional substeps, see FIG. 6E. Step 612c1 fits the MBP derived from the second database into quadratic curves parameterized by age, for each gender. Step 612c2 fits the PWV Arm derived from the third database into a quadratic curve parameterized by age. Step 612c3 fits the PWV_{Central} derived from the first database into a quadratic curve parameterized by age. Step 612c4 fits the Slope_{Central} derived from the first database into a quadratic curve parameterized by age. Note: Steps 612c2 through 612c4 are the same as Steps 612a2 through 612a4, and may only be performed once for SBP, DBP, and MBP. Step 612c5 finds the MBPXfrm intercept point (bₘ) and slope (mₘ) as follows:

$$MBPXfrm = [ {b}_{m} , {m}_{m} ] \text{:} wherein$$

$${m}_{m} = {Slope}_{Central} ( age ) \times \frac{{PWV}_{Arm} ( age )}{{PWV}_{Central} ( age )}$$

$${b}_{m} = MBP ( age , gender ) - {m}_{m} \times {PWV}_{Arm} ( age ) .$$

A system and method have been provided for deriving pseudo-calibrated PWV-BP transforms from demographic data. Examples of particular statistical processes have been presented to illustrate the invention. However, the invention is not limited to merely these examples. Other variations and embodiments of the invention will occur to those skilled in the art.

We claim:

1. A method for using demographic data to derive a pulse wave velocity-blood pressure (PWV-BP) transform, the method comprising:
providing a PWV measurement device comprising a non-transitory memory, processor, and a calibration application enabled as a sequence of processor executable steps for providing pseudo-calibrated PWV values;
loading into the memory a first database cross-referencing age and central-aortic PWV-BP transforms;
loading into the memory a second database cross-referencing age and gender with systolic and diastolic blood pressure;
loading into the memory a third database cross-referencing age and whole-arm PWV, where whole-arm PWV measures a distance between a superasternal notch and index finger, divided by a transit time of an arterial pulse from to heart to the index finger tip;
accepting age and gender data from a first user;
the calibration application interpolating the information from the first, second, and third databases, and deriving a pseudo-calibrated whole-arm PWV-BP transform; and,
supplying the pseudo-calibrated whole-arm PWV-BP transform for the first user.

2. The method of claim 1 wherein interpolating the information from the first, second, and third databases includes:
determining a systolic blood pressure transform (SBPXfrm) incorporating the systolic blood pressure (SBP) derived from the second database, the whole-arm PWV (PWV_{Arm}) derived from the third database, the central aortic PWV (PWV_{Central}) derived from the first database, and a transform slope (Slope_{Central}) derived from the first database;
determining a diastolic blood pressure transform (DBPXfrm) incorporating the diastolic blood pressure (DBP) derived from the second database, the whole-arm PWV (PWV_{Arm}) derived from the third database, the central aortic PWV (PWV_{Central}) derived from the first database, and the transform slope (Slope_{Central}) derived from the first database; and,
determining a mean blood pressure transform (MBPXfrm) incorporating a mean blood pressure (MBP) derived from the second database, the whole-arm PWV (PWV_{Arm}) derived from the third database, the central aortic PWV (PWV_{Central}) derived from the first database, and the transform slope (Slope_{Central}) derived from the first database.

3. The method of claim 2 wherein determining the SBPXfrm includes:
fitting the SBP data derived from the second database into quadratic curves parameterized by age, for each gender;
fitting the PWV_{Arm} derived from the third database into a quadratic curve parameterized by age;
fitting the PWV_{Central} derived from the first database into a quadratic curve parameterized by age; and,
fitting the Slope_{Central} derived from the first database into a quadratic curve parameterized by age.

4. The method of claim 3 wherein determining the SBPXfrm includes finding an intercept point and slope as follows:
$$SBPXfrm = [ {b}_{s} , {m}_{s} ] wherein$$
$${m}_{s} = {Slope}_{Central} ( age ) \times \frac{{PWV}_{Arm} ( age )}{{PWV}_{Central} ( age )}$$
$${b}_{s} = SBP ( age , gender ) - {m}_{S} \times {PWV}_{Arm} ( age ) .$$

5. The method of claim 2 wherein determining the DBPXfrm includes:
fitting the DBF derived from the second database into a quadratic curve parameterized by age, for each gender;
fitting the PWV_{Arm} derived from the third database into a quadratic curve parameterized by age;
fitting the PWV_{Central} derived from the first database into a quadratic curve parameterized by age; and,
fitting the Slope_{Central} derived from the first database into a quadratic curve parameterized by age.

6. The method of claim 5 wherein determining the DBPXfrm includes finding an intercept point and slope as follows:
$$DBPXfrm = [ {b}_{d} , {m}_{d} ] . wherein$$
$${m}_{d} = {Slope}_{Central} ( age ) \times \frac{{PWV}_{Arm} ( age )}{{PWV}_{Central} ( age )}$$
$${b}_{d} = DBP ( age , gender ) - {m}_{d} \times {PWV}_{Arm} ( age ) ;$$

7. The method of claim 2 wherein determining the MBPXfrm includes:
fitting the MBP derived from the second database into quadratic curves parameterized by age, for each gender;
fitting the PWV_{Arm} derived from the third database into a quadratic curve parameterized by age;
fitting the PWV_{Central} derived from the first database into a quadratic curve parameterized by age; and,
fitting the Slope_{Central} derived from the first database into a quadratic curve parameterized by age.

8. The method of claim 7 wherein determining the MBPXfrm includes finding an intercept point and slope as follows:
$$MBPXfrm = [ {b}_{m} , {m}_{m} ] \text{:} wherein$$
$${m}_{m} = {Slope}_{Central} ( age ) \times \frac{{PWV}_{Arm} ( age )}{{PWV}_{Central} ( age )}$$
$${b}_{m} = MBP ( age , gender ) - {m}_{m} \times {PWV}_{Arm} ( age ) .$$

9. The method of claim 1 further comprising;
measuring the whole-arm PWV of the first user;
using the pseudo-calibrated whole-arm PWV-BP transform to derive a blood pressure associated with the first user whole-arm PWV measurement.

10. The method of claim 1 wherein the first database cross-references patient age to central-aortic PWV-BP transforms;
wherein the second database cross-references patient age to SBP and DBP;
wherein the third database cross-references age to whole-arm PWV;
wherein accepting the age data from the first user includes accepting a specific date of birth; and,
wherein supplying the pseudo-calibrated PWV-BP transform for the first user includes supplying a pseudo-calibrated PWV-BP transform based upon a current date and the first user's date of birth.

11. A system using demographic data to derive a pulse wave velocity-blood pressure (PWV-BP) transform, the system comprising:
a PWV measurement interface comprising an electrocardiogram (ECG) sensor and a photoplethysmography (PPG) sensor for respectively measuring first user ECG and PPG signals;
a processor;
a user interface (UI) to accept age information from the first user, and to supply a pseudo-calibrated blood pressure (BP) value;
a non-transitory memory;
a calibration application embedded in the memory and enabled as a sequence of processor executable steps, the calibration application accepting the ECG and PPG signals, the first user age, and information interpolated from a first database, a second database, and a third database, to calculate the pseudo-calibrated BP value for the first user;
wherein the first database of information cross-references age compared to central-aortic PWV-BP transforms;
wherein the second database of information cross-references age and gender compared to systolic and diastolic blood pressure; and,
wherein the third database of information cross-references age as compared to whole-arm PWV, where whole-arm PWV measures a distance between a superasternal notch and index finger, divided by a transit time of an arterial pulse from to heart to the index finger tip.

12. The system of claim 11 wherein the UI accepts first user gender information; and,
wherein the calibration application calculates the pseudo-calibrated BP value in response to the first user gender,

13. The system of claim 12 wherein the first, second, and third databases reside in the memory; and,
wherein the calibration application determines a systolic blood pressure transform (SBPXfrm) incorporating the systolic blood pressure (SBP) derived from the second database, the whole-arm PWV (PWV_{Arm}) derived from the third database, the central aortic PWV (PWV_{Central}) derived from the first database, and a transform slope (Slope_{Central}) derived from the first database;
wherein the calibration application determines a diastolic blood pressure transform (DBPXfrm) incorporating the diastolic blood pressure (DBP) derived from the second database, the whole-arm PWV (PWV_{Arm}) derived from the third database, the central aortic PWV (PWV_{Central}) derived from the first database, and the transform slope (Slope_{Central}) derived from the first database; and,
wherein the calibration application determines a mean blood pressure transform (MBPXfrm) incorporating a mean blood pressure (MBP) derived from the second database, the whole-arm PWV (PWV_{Arm}) derived from the third database, the central aortic PWV (PWV_{Central}) derived from the first database, and the transform slope (Slope_{Central}) derived from the first database.

14. The system of claim 13 wherein the calibration application determines the SBPXfrm by:
fitting the SBP data derived from the second database into quadratic curves parameterized by age, for each gender;
fitting the PWV_{Arm} derived from the third database into a quadratic curve parameterized by age;
fitting the PWV_{Central} derived from the first database into a quadratic curve parameterized by age; and,
fitting the Slope_{Central} derived from the first database into a quadratic curve parameterized by age.

15. The system of claim 12 wherein the calibration application determines a SBPXfrm intercept point and slope as follows:
$$SBPXfrm = [ {b}_{s} , {m}_{s} ] wherein$$
$${m}_{s} = {Slope}_{Central} ( age ) \times \frac{{PWV}_{Arm} ( age )}{{PWV}_{Central} ( age )}$$
$${b}_{s} = SBP ( age , gender ) - {m}_{S} \times {PWV}_{Arm} ( age ) ;$$
wherein Slope_{Central}(age) is a quadratic model of central aortic PWV-BP slope as a function of age:
wherein PWV_{Arm}(age) is a quadratic model of whole-arm PWV as a function of age;
wherein PWV_{Central}(age) is a quadratic model of central aortic PWV as a function of age; and,
wherein SBP(age, gender) is a quadratic model of systolic blood pressure as a function of age and gender.

16. The system of claim 13 wherein the calibration application determines the DBPXfrm by:
fitting the DBP derived from the second database into a quadratic curve parameterized by age, for each gender;
fitting the PWV_{Arm} derived from the third database into a quadratic curve parameterized by age;
fitting the PWV_{Central} derived from the first database into a quadratic curve parameterized by age; and,
fitting the Slope_{Central} derived from the first database into a quadratic curve parameterized by age.

17. The system of claim 12 wherein the calibration application determines a DBPXfrm intercept point and slope as follows:
$$DBPXfrm = [ {b}_{d} , {m}_{d} ] . wherein$$
$${m}_{d} = {Slope}_{Central} ( age ) \times \frac{{PWV}_{Arm} ( age )}{{PWV}_{Central} ( age )}$$
$${b}_{d} = DBP ( age , gender ) - {m}_{d} \times {PWV}_{Arm} ( age ) ;$$
wherein Slope_{Central}(age) is a quadratic model of central aortic PWV-BP slope as a function of age:
wherein PWV_{Arm}(age) is a quadratic model of whole-arm PWV as a function of age;
wherein PWV_{Central}(age) is a quadratic model of central aortic PWV as a function of age; and,
wherein DBP(age, gender) is a quadratic model of systolic blood pressure as a function of age and gender.

18. The system of claim 13 wherein the calibration application determines the MBPXfrm by:
fitting the MBP derived from the second database into quadratic curves parameterized by age, for each gender;
fitting the PWV_{Arm} derived from the third database into a quadratic curve parameterized by age;
fitting the PWV_{Central} derived from the first database into a quadratic curve parameterized by age; and,
fitting the Slope_{Central} derived from the first database into a quadratic curve parameterized by age.

19. The system of claim 12 wherein the calibration application determines a MBPXfrm intercept point and slope as follows:
$$MBPXfrm = [ {b}_{m} , {m}_{m} ] \text{:} wherein$$
$${m}_{m} = {Slope}_{Central} ( age ) \times \frac{{PWV}_{Arm} ( age )}{{PWV}_{Central} ( age )}$$
$${b}_{m} = MBP ( age , gender ) - {m}_{m} \times {PWV}_{Arm} ( age ) ;$$
wherein Slope_{Central}(age) is a quadratic model of central aortic PWV-BP slope as a function of age:
wherein PWV_{Arm}(age) is a quadratic model of whole-arm PWV as a function of age;
wherein PWV_{Central}(age) is a quadratic model of central aortic PWV as a function of age; and,
wherein MBP(age, gender) is a quadratic model of systolic blood pressure as a function of age and gender.

20. The system of claim 12 wherein the UI accepts a first user specific date of birth; and,
wherein the calibration application calculates the pseudo-calibrated BP value based upon a current date, the first user's date of birth, and quadratic models of PWV-BP transform slope, PWV, and blood pressure for the first user's date of birth.
